(19) United States
(12) Patent Application Publication
(10) Pub. No.: US 2002/0068892 A1
(43) Pub. Date: Jun. 6, 2002

(54) Muff vest apron arm support

(76) Inventor: Alberta S. Estep, Mendenhall, MS (US)

Correspondence Address: Alberta S Estep, 402 Old Gravel 49, Mendenhall, MS, 39114, US

(21) Appl. No.: 09/737,285

(22) Filed: Dec. 4, 2000

(51) Int. Cl. A61F 13/00

(52) U.S. Cl. 602/60

(57) ABSTRACT

A universal nonrestrictive arm support that utilizes the torso of the body to distribute the weight of the arm using an attached muff placed on the front part of the wrap around vest apron thus allowing the wearer to care for their disabled arm with the able arm.

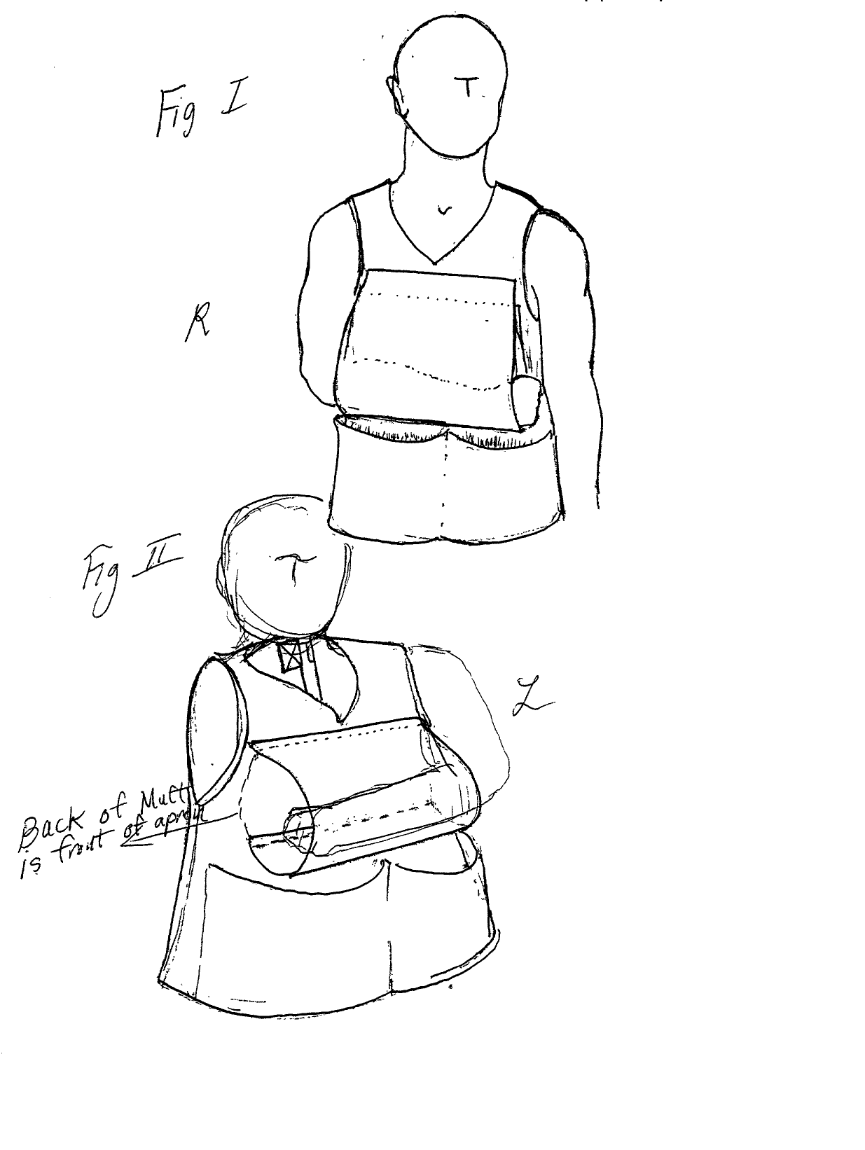

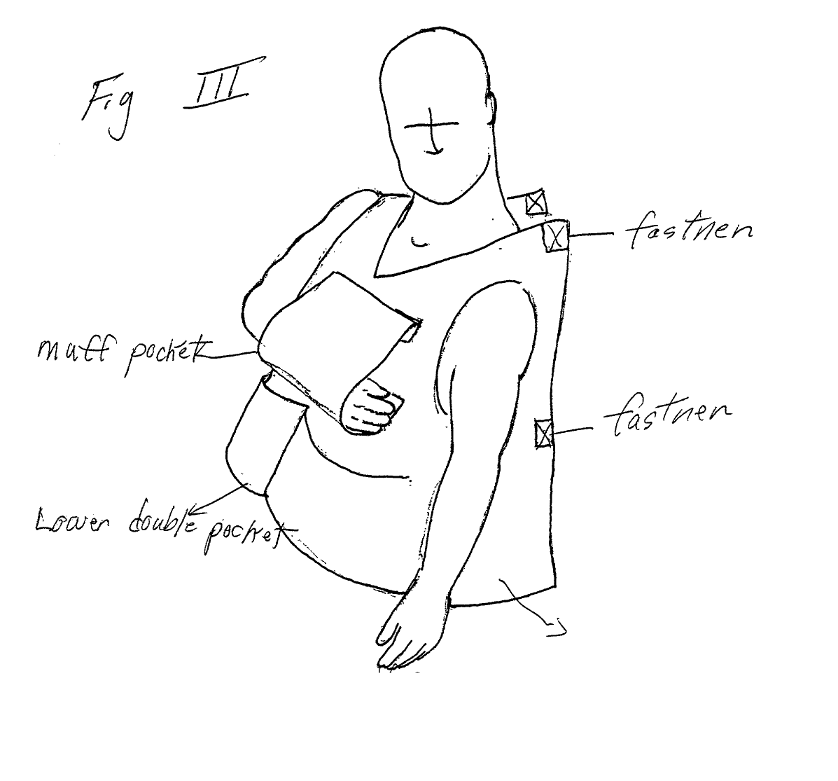

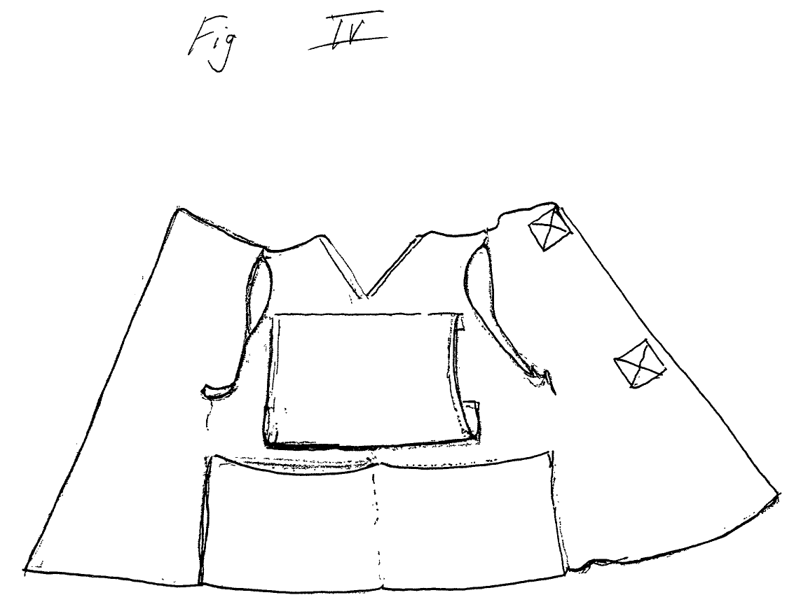

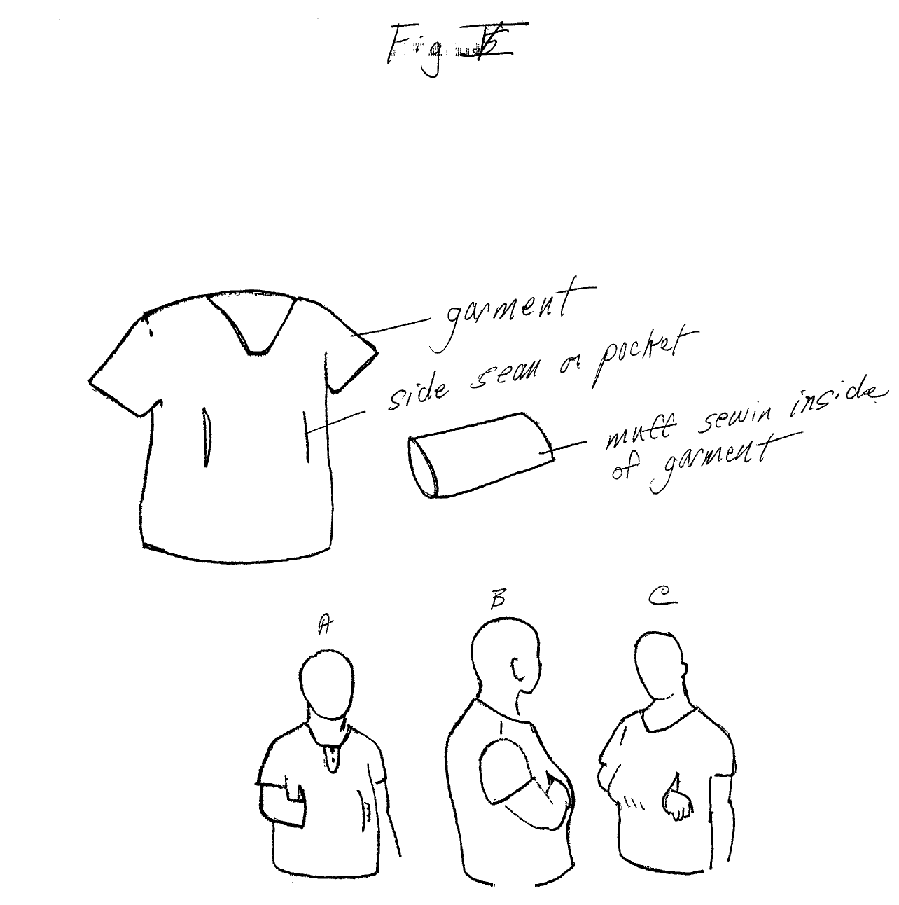

### BACKGROUND INFORMATION

The invention is a new and improved way of using an arm support in a nonrestrictive way. All other type garments or slings restrict or immobilize the arm. There is a need for a new and improved arm support that doesn't restrict movement of the arm. The universal nonrestrictive muff vest apron is needed in the field of medicine. The invention is needed to care for the arm in a nonrestrictive manner. The invention is especially useful for hemiplegics or those who have lost the use of either arm. The invention is needed when the care of the arm falls on the wearer. The invention is an improvement to existing arm support. It is unique in a way that it forms the foundation for support by the torso which allows the weight to be evenly distributed with no pull on the neck. The muff pocket in front allows the wearer to be in control of his or her movements. The garment is an improvement that considers all other arm supports have served their purpose.

### SUMMARY

The Muff Vest Apron is a uniquely designed support garment. It is a nonrestrictive garment. It is a unique way to use the apron vest along with the muff pocket to form a complete garment that is nonrestrictive to the wearer.

### DESCRIPTION

### The Muff Vest Apron Arm Support

FIG. I Shows the garment being worn by the patient thus showing there is no restriction of the arm to the wearer. The muff is permanently attached to the apron allowing the front part of the vest to be the back side of muff, to form a complete tubular pocket.

FIG. II A reverse use if the muff vest apron, from one arm to the other. One would be able to reach through the open end muff to retrieve an arm without restriction.

FIG. III Shows that the apron is wrapped around the torso with back fasteners. The garment can be put on or removed without any restrictions. The fastener can be accessed easily by the able arm.

FIG. IV Shows the complete garment with all step to invention including the lower patch pockets used for transporting needed items. All other support garments are serving their purpose.

FIG. V Muff Pocket Support can be attached to an existing garment by being placed inside with side pocket slits.

1. The invention claims to be a universal nonrestrictive arm support. The arm is supported from the wrap around vest apron forming the foundation for support by the torso of the body. By supporting the disabled arm from a front muffpocket. The invention allows the wearer the freedom of caring for the disabled arm by the able arm.
